(19) United States
(12) Patent Application Publication
(10) Pub. No.: US 2023/0256146 A1
(43) Pub. Date: Aug. 17, 2023

(54) SYSTEM, METHODS, AND APPARATUS HAVING A CIRCULAR BUFFER FOR THE REPLAY OF RENAL THERAPY MACHINE ALARMS AND EVENTS

(71) Applicant: Gambro Lundia AB, Lund (SE)

(72) Inventors: Gerard PRINDLE, Shoreview, MN (US); Andrew HODGES, St Louis Park, MN (US); Klaus OBERGFELL, Excelsior, MN (US); Bernd WITTNER, Minneapolis, MN (US); Rikard HULT, Lomma (SE)

(21) Appl. No.: 18/016,231

(22) PCT Filed: Jul. 8, 2021
(86) PCT No.: PCT/EP2021/069007

§ 371 Date: Jan. 13, 2023

Related U.S. Application Data
(60) Provisional application No. 63/053,201, filed on Jul. 17, 2020.

(51) Int. Cl. A61M 1/16; G16H 40/63; A61M 5/172

(52) U.S. Cl. CPC A61M 1/1601; G16H 40/63; A61M 5/172; G06F 12/0802

(57) ABSTRACT

A system, methods, and apparatus having a circular buffer for the reply of renal therapy machine alarms and events are disclosed. An example renal therapy apparatus includes a therapy operations processor configured to generate alarms, events, and high fidelity medical device data. The renal therapy apparatus also includes a memory device having a circular buffer configured to store a duration of medical device data. The renal therapy apparatus further includes a control processor configured to receive a stream of medical device data from the therapy operations processor, and write the stream to the circular buffer such that a most recent duration of the stream is stored. The control processor is also configured to detect an occurrence of an alarm or event, and create a reply record that includes an identifier of the alarm or event and the medical device data that is stored in the circular buffer.

### BACKGROUND

For many medical devices, there exists a data disconnect. Through improvements in processing, known medical devices, including renal therapy machines and infusion pumps, generate significant amounts of medical device data. These devices contain components, such as pumps, sensors, motors, valves, dialyzers, etc., which each provide medical device data regarding detected state, status, faults, or measurements. Additionally, known medical devices have a therapy processor that tracks parameters and treatment status for a medical treatment including, for example, a hemodialysis treatment type, dialysis fluid dextrose level, volume of fluid administered to a patient, treatment time, estimated ultrafiltration (“UF”) removed from a patient, detected alarms, etc. Oftentimes, this data is generated at a rate between 2 Hz to 100 Hz and provides a precise picture of an operating state of a medical device. However, while the medical device can easily create all this data, it cannot easily store or transmit all this data.

To store all the medical device data generated during a treatment, a medical device would need terabytes of hard disk space. A known issue with many medical devices is that internal memory (e.g., a machine log) is not sufficient to store all the generated data. Instead, to conserve memory or hard disk space (and corresponding cost), medical devices may only record data every five seconds to two minutes. In some instances, known medical devices only store data when there is a change in a data value. This reduced amount of stored medical device data permits usage of smaller hard disks that are only a small fraction of the capacity needed if all the medical device data was stored.

In addition to memory constraints, network transmission constraints also limit the amount of data that is used. It is generally not feasible for medical devices to transmit medical device data at a rate between 2 Hz to 100 Hz. Over the course of a treatment, which could amount to terabytes of data transmitted. Many medical facilities in addition to patient homes (for home-based treatments) do not have the network bandwidth for transmitting such a large amount of medical device data. Instead, known medical devices transmit medical device data every five seconds to two minutes, or wait until a treatment has ended before transmitting stored medical device data that was recorded at five second to two minute intervals.

Oftentimes, the stored medical device data does not provide enough resolution to enable faults, alarms, or other events to be diagnosed. In many instances, significant component responses occur between the time intervals for storing medical device data. Additionally, alarm conditions can develop quickly between the data storage time intervals. As a result, critical data for identifying a cause of an alarm or other event is missing, thereby making it more difficult for modeling, diagnosis, and correction.

A need accordingly exists for a medical device that provides higher resolution medical device data while also conserving memory usage and network bandwidth consumption.

### SUMMARY

Example systems, methods, and apparatus are disclosed herein that provide one or more circular buffer in a medical device, such as a renal therapy machine or an infusion pump. The circular buffer(s) is configured to record medical device data at a rate the data is generated, referred to herein as high resolution or high fidelity data. Instead of filling a memory device, the circular buffer(s) overwrites relatively old medical device data with most recent medical device data. When a specified alarm or event condition occurs, the medical device is configured to transmit or otherwise create a record that contains the high-resolution data in the circular buffer(s).

The circular buffer(s) (e.g., memory device(s) including or configured as circular buffer(s)) is configured to record medical device data for providing a sufficient data history for root-cause diagnosis regarding a detected alarm or event condition. In some instances, a circular buffer may store thirty seconds of high fidelity data, one minute of high fidelity data, two minutes of high fidelity data, etc. Additionally, in some instances, a circular buffer may store medical device data for a certain time duration after an alarm or event condition to provide a snapshot of the medical device before and after the condition. This post-alarm/event time duration may be as little as half a second, two seconds, ten seconds, thirty seconds, etc. Further, in some instances, the time duration may be zero seconds, where only medical device data before and at the time of an alarm or event condition is stored.

In some embodiments, the medical device may include more than one circular buffer that is located in one or more memory devices. Each circular buffer may be configured to receive medical device data that is generated one data rate (e.g., 60 Hz, 20 Hz, 10 Hz, etc.). Additionally or alternatively, each circular buffer may be configured to receive medical device data that is relevant for a specified alarm or event condition. When an alarm or event condition is satisfied, the medical device accesses the medical device data from the appropriate circular buffer(s). This enables only the medical device data that is relevant for the detected alarm or event condition to be analyzed.

Further, in some embodiments, the example system, method, and apparatus are configured to use models that provide a representation or snapshot of a medical device when an alarm or event condition is triggered. The models consume certain relevant medical device data that is related to an alarm or event condition. For example, a condition related to an alarm for a fluid line occlusion in a dialysis fluid line of a hemodialysis machine may be associated with medical device data including fluid flow rate, fluid pressure, therapy state, and measurements from leak detection sensors. The system, method, and apparatus model the associated medical device data stored in the circular buffer designated for the occlusion alarm condition. The system, method, and apparatus then display the models of the flow rate, fluid pressure, therapy state, and leak detection measurements to enable a user to diagnosis a cause of the occlusion alarm condition. In some instances, the system, method, and apparatus may analyze the data using slope analysis, derivative analysis, template matching, threshold analysis, etc., to identify or provide a recommendation regarding a cause of the occlusion alarm condition. An operator uses the displayed information to correct for the condition for the current treatment and/or future occurrences of a similar treatment.

In addition to providing diagnostics at the medical device, the example system, method, and apparatus are configured to transmit a record of medical device data to a remote server via a network. The record includes an identifier of an alarm or event condition in addition to the corresponding medical device data in a related circular buffer. The server may include one or more algorithm that enables the received data to be modeled and analyzed to re-create the situation that triggered the detected alarm or event condition for root-cause diagnosis. Further, the server may refine root-cause analysis models, which may be transmitted to the medical device to improve local diagnosis.

The example methods, apparatus, and system that use the circular buffer disclosed herein operate with any type of medical device or machine that is applicable, for example, to fluid delivery for plasmapherisis, hemodialysis (“HD”), hemofiltration (“HF”) hemodiafiltration (“HDF”), and continuous renal replacement therapy (“CRRT”) treatments. The methods, apparatus, and system described herein is also applicable to peritoneal dialysis (“PD”), intravenous drug delivery, and nutritional fluid delivery. The above different treatment modalities and associated devices may be referred to herein collectively or generally individually as medical fluid delivery or treatment and associated devices or machines.

The above modalities may be provided by a medical fluid delivery machine (e.g., a medical device) that houses components needed to deliver medical fluid, such as one or more pumps, valves, heaters if needed, online medical fluid generation equipment if needed, sensors, such as any one, or more, or all of pressure sensors, conductivity sensors, temperature sensors, air detectors, blood leak detectors, and the like, user interfaces, and control units, which may employ one or more processors and memory to control the above-described equipment. The medical fluid delivery machine may also include one or more filter, such as a dialyzer or hemofilter, for cleansing blood and/or an ultrafilter for purifying water, dialysis fluid, or other treatment fluid.

The methods, apparatus, and system and medical fluid delivery machine described herein may be used with home-based machines. For example, the systems may be used with home HD, HF, HDF or PD machines, which are operated at the patient's convenience. One such home system is described in U.S. Pat. No. 8,029,454 (“the '454 Patent”), issued Oct. 4, 2011, entitled “High Convection Home Hemodialysis/Hemofiltration And Sorbent System”, filed Nov. 4, 2004, assigned to the assignees of the present application. Other such home systems are described in U.S. Pat. No. 8,393,690 (“the '690 Patent”), issued Mar. 12, 2013, entitled “Enclosure for a Portable Hemodialysis System”, filed Aug. 27, 2008. The entire contents of each of the above references are incorporated herein by reference and relied upon.

Aspects of the subject matter described herein may be useful alone or in combination with one or more other aspect described herein. Without limiting the foregoing description, in a first aspect of the present disclosure, a medical device (104), in particular a renal therapy apparatus, is provided comprising a memory device (111) configured as a circular buffer (302; 502); therapy module (107) configured to generate (i) alarms, (ii) events, and (iii) medical device data (304), (i), (ii), and (iii) being related to operation of the medical device (104), in particular the renal therapy apparatus for performing a medical treatment, in particular a renal therapy treatment, the medical device data including at least two of first data generated at a first Hz rate, for example 1 Hz rate, second data generated at a second Hz rate, for example 5 Hz rate, third data generated at a third Hz rate, for example 10 Hz rate, fourth data generated at a fourth Hz rate, for example 20 Hz rate, fifth data generated at a fifth Hz rate, for example 60 Hz rate, and sixth data generated at an asynchronous rate, wherein first Hz rate, second Hz rate, third Hz rate, fourth Hz rate, and fifth Hz rate are different from each other; a control processor (109) communicatively coupled to the memory device (111) and the therapy module (107), the control processor (109) configured to receive the medical device data (304) at the specified data rates from the therapy module (107), store a first stream of the medical device data (304) to the memory device (111) in a circular buffer configuration such that previous medical device data stored to the memory device (111) is overwritten after being in the memory device (111) for a first specified time duration, determine that at least one of an alarm or an event has been generated by the therapy module (107), store, for a second specified time duration after the occurrence of the at least one of the alarm or the event, a second stream of the medical device data (304) to the memory device (111) in the circular buffer configuration such that previous medical device data stored to the memory device (111) is overwritten after being in the memory device (111) for the first specified time period, and after the second specified time duration has elapsed, create a file record (406) that includes the medical device data (304) contained in the circular buffer (302; 502) and information indicative of the at least one of the generated alarm or event.

In a second aspect according to the previous aspect, the medical device data (304) includes at least one of pump rate data, pressure data, temperature data, scale data, or diagnostic data.

In a third aspect according to any one of the previous aspects, the first specified time duration is between ten seconds and two minutes and the second time duration is between zero seconds and thirty seconds.

In a fourth aspect according to any one of the previous aspects, the device further comprises a port (202) to receive a portable memory device (204), wherein the control processor (109) is configured to transmit the file record (406) to the portable memory device (204) after detecting that the portable memory device (204) is communicatively coupled to the port.

In a fifth aspect according to any one of the previous aspects, the control processor (109) is configured to transmit the file record (406) to an EMR server (108) via a medical network (106) after the file record (406) is created.

In a sixth aspect according to any one of the previous aspects, the file record (406) includes at least a portion of the first stream of the medical device data (304) before the at least one of the alarm or the event and the second stream of the medical device data (304) after the at least one of the alarm or the event to enable a server to recreate conditions of the renal therapy apparatus for identifying a cause of the at least one of the alarm or the event.

In a seventh aspect a medical device (104) is provided, optionally according to any one of the previous aspects, comprising a therapy module (107) configured to generate alarms, events, and high fidelity medical device data that are related to an operation of the medical device (104) for performing a medical treatment; a memory device (111) including a first circular buffer (502a) configured to store a first duration of medical device data (304) and a second circular buffer (502b) configured to store a second duration of medical device data (304), the first circular buffer (502a) configured to store a first subset of the medical device data (304) and the second circular buffer (502b) configured to store a second subset of the medical device data (304); a control processor (109) communicatively coupled to the memory device (111) and the therapy module (107), the control processor (109) configured to receive a stream of medical device data (304) from the therapy module (107), identify as a first stream, the first subset of the received medical device data (304), identify as a second stream, the second subset of the received medical device data (304), write the first stream to the first circular buffer such that a most recent first duration of the first stream is stored, write the second stream to the second circular buffer such that a most recent second duration of the second stream is stored, detect an occurrence of an alarm or event, and create a record (406) that includes an identifier of the alarm or event and at least the first subset of the medical device data (304) that is stored in the first circular buffer (502a).

In an eighth aspect according to previous aspect 7, the control processor (109) is configured to additionally include in the record (406) the second subset of the medical device data that is stored in the second circular buffer (502b).

In a ninth aspect according to any one of the previous aspects 7 and 8, the first duration and the second duration have a same time duration.

In a tenth aspect according to any one of the previous aspects 7 to 9, the first duration has a time duration that is longer than the second duration.

In an eleventh aspect according to any one of the previous aspects 7 to 10, the first duration and the second duration are each between ten seconds and two minutes.

In a twelfth aspect according to any one of the previous aspects 7 to 11, the first subset of the medical device data (304) is generated at a first data rate and the second subset of medical device data (304) is generated at a second, different data rate.

In a thirteenth aspect according to aspect 12, the first data rate and the second data rate are each between 1 Hz and 100 Hz.

In a fourteenth aspect according to any one of the previous aspects 12 and 13, at least one of the first data rate or the second data rate includes an asynchronous data rate.

In a fifteenth aspect according to any one of the previous aspects 7 to 14, the detected alarm or event is a first alarm or event, and wherein the first subset of the medical device data (304) is associated with the first alarm or event and the second subset of the medical device data (304) is associated with a second, different alarm or event.

In a sixteenth aspect according to any one of the previous aspects 7 to 15, the alarm or event includes at least one of an occlusion alarm, a pressure alarm, a low fluid volume alarm, a flow rate alarm, a syringe alarm, a fluid leak detection alarm, a blood leak detection alarm, an air bubble detection alarm, a power supply alarm, a treatment pause event, a treatment stoppage event, or a treatment parameter change event.

In a seventeenth aspect according to any one of the previous aspects 7 to 16, the control processor (109) is configured to transmit the record (406) via at least one of (i) a network (106) to a server (108), or (ii) a port (202) to a portable memory device (204) for diagnosis of a cause of the alarm or event.

In an eighteenth aspect according to any one of the previous aspects 7 to 17, the control processor (109) is configured to model or analyze at least the first subset of the medical device data (304) that is included within the record (406) for diagnosis of a cause of the alarm or event.

In a nineteenth aspect according to any one of the previous aspects 7 to 18, the control processor (109) is configured to display on a display screen in a time-series graph at least some of the first subset of the medical device data (304) included within the record (406).

In a twentieth aspect according to any one of the previous aspects 7 to 19, the medical device (104) includes at least one of a renal therapy machine or an infusion pump.

In a twenty-first aspect according to any one of the previous aspects, the device comprises one or more sensors and one or more actuators including one or more pumps, wherein the therapy module (107) is configured to receive data from the one or more sensors and to control the one or more actuators to perform a medical task.

In a twenty-second aspect according to any one of the previous aspects, the medical device (104) is a renal therapy apparatus for the treatment of renal disease with extracorporeal circulation of blood comprising: a filtration unit having a primary chamber and a secondary chamber separated by a semi-permeable membrane; a blood circuit comprising: a blood withdrawal line extending between a first end connected to an inlet of the primary chamber and a second end for connection to a patient; and a blood return line extending between a first end connected to an outlet of the primary chamber and a second end for connection to said patient; a blood pump to circulate blood in the blood circuit; a dialysate line connected to an outlet of the secondary chamber; optionally, one or more lines to transfer a respective solution into blood.

In a twenty-third aspect a medical device method is provided for a medical device, optionally according to any one of the previous aspects, comprising: receiving, in a control processor (109) of the medical device, a stream of medical device data (304); identifying, via the control processor (109), as a first stream, a first subset of the received medical device data (304); identifying, via the control processor (109), as a second stream, a second subset of the received medical device data (304); writing, via the control processor (109), the first stream to a first circular buffer (502a) in a memory device (111) such that a most recent first duration of the first stream is stored; writing, via the control processor (109), the second stream to a second circular buffer (502b) in the memory device (111) such that a most recent second duration of the second stream is stored; receiving, in the control processor (109), an indication or an alarm or an event; and creating, via the control processor (109), a record (406) that includes an identifier of the alarm or event and at least the first subset of the medical device data (304) that is stored in the first circular buffer (502a).

In a twenty-fourth aspect according to the previous aspect, creating the record (406) further comprises including the second subset of the medical device data (304) that is stored in the second circular buffer (502b).

In a twenty-fifth aspect according to any one of the previous aspects 23 and 24, the first circular buffer (502a) is configured to receive medical device data (304) that is generated at a first data rate and the second circular buffer (502b) is configured to receive medical device data (304) that is generated at a second, different data rate.

In a twenty-sixth aspect according to the previous aspect 25, the first duration is different from the second duration and each of the first and second durations are between ten seconds and two minutes.

In accordance with a twenty-seventh aspect of the present disclosure, any of the structure and functionality illustrated and described in connection with FIGS. 1 to 13 may be used in combination with any of the structure and functionality illustrated and described in connection with any of the other of FIGS. 1 to 13 and with any one or more of the preceding aspects.

In light of the present disclosure and the above aspects, it is therefore an advantage of the present disclosure to provide a temporary memory for high fidelity or high resolution medical device data.

It is another advantage of the present disclosure to provide a snapshot of high fidelity or high resolution medical device data that relates to a detected alarm or event condition in a medical device.

It is further advantage of the present disclosure to model or analyze a snapshot of high fidelity or high resolution medical device data that is associated with a detected alarm or event condition to diagnosis a cause of the condition.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a diagram of a medical device environment including a medical device having at least one circular buffer, according to an example embodiment of the present disclosure.

FIG. 2 is another diagram of the medical device environment, according to an example embodiment of the present disclosure.

FIGS. 3 and 4 are diagrams of an example procedure for using a circular buffer for creating records or files of medical device data that relates to an event or an alarm, according to an example embodiment of the present disclosure.

FIGS. 5 and 6 are diagrams of an example procedure for using multiple circular buffers for creating records or files of medical device data that relates to an event or an alarm, according to an example embodiment of the present disclosure.

FIG. 7 is a diagram of a graph that is illustrative of medical device data stored in a circular buffer and written to a record for diagnosis of an event or alarm, according to an example embodiment of the present disclosure.

FIG. 8 is a diagram of a graph that is illustrative of medical device data generated by the medical device of FIGS. 1 and 2, according to an example embodiment of the present disclosure.

FIG. 9 is a diagram of a graph that shows which data is available without the use of circular buffers, according to an example embodiment of the present disclosure.

In contrast FIG. 10 is a diagram of a graph that shows which portion of medical device data is available with circular buffers, according to an example embodiment of the present disclosure.

FIGS. 11 and 12 are diagrams of a user interface with diagnosis information provided by a control processor and/or a management server, according to an example embodiment of the present disclosure.

FIG. 13 is a diagram of a diagnosis process, according to an example embodiment of the present disclosure.

### DETAILED DESCRIPTION

Methods, systems, and apparatus are disclosed herein that use circular buffers to enable the replay of conditions of medical devices during the generation of alarms or events. The methods, systems, and apparatus are configured to record a certain time duration of medical device data, which is stored in one or more circular buffers. The length of the time duration is specified so as to provide sufficient resolution to provide a snapshot of a condition of a medical device when an alarm or an event is created.

Reference is made herein to circular buffers. As disclosed herein, a circular buffer, also known as a ring buffer or a cyclic buffer, is a data structure having a fixed-size. In the context disclosed herein, the fixed size of the buffer corresponds to a time duration for receiving medical device data that is sufficient to enable a replay of medical device conditions leading up to (and after) detection of an alarm or an event. The time duration may be set to a value between ten seconds and two minutes, for example. High fidelity medical device data is written to the circular buffer until the buffer is filled. At this point, newly received medical device data is written over the data at the end of the buffer, which corresponds to the relatively oldest data. The process is repeated as new medical device data is received such that the buffer does not contain data that is older than the specified time duration.

Reference is also made herein to medical treatment data and/or medical event data (collectively referred to as medical device data herein). The medical device data includes data generated at a medical device and data received from one or more sensors (e.g., a blood pressure sensor, a weigh scale, a blood gas analyzer, etc.) that are communicatively coupled to a medical device. The medical device data may be generated by any medical device including a peritoneal dialysis machine, a critical care dialysis machine, a continuous renal replacement therapy (“CRRT”) machine, a hemodialysis machine, a water preparation/purification device, a nutrition compounding machine, an infusion pump, etc. The medical device data may be in a JavaScript Object Notation (“JSON”) format, a HyperText Markup Language (“HTML”) format, an Extensible Markup Language (“XML”) format, a comma-separated values (“CSV”) format, a text format, and/or a Health-Level-7 (“HL7”) format.

The medical device data includes treatment programming information. Treatment programming information includes one or more parameter that define how a medical device is to operate to administer a treatment to a patient. For a CRRT therapy, the parameters may specify a flow rate of fresh dialysis fluid, a total flow volume (or volume per bag) of dialysis fluid, a dialysis fluid concentration, a total number of fresh dialysis bags connected to a CRRT medical device, a target UF removal, a blood flow rate, a total number of drain/effluent bags connected, hemofilter or dialyzer information, a replacement fluid volume and/or flow rate, and/or an amount (and/or rate) of heparin or other pharmaceutical/additive that is to be added to a patient's recirculated blood.

For a peritoneal dialysis therapy, the parameters may specify an amount (or rate) of fresh dialysis fluid to be pumped into a peritoneal cavity of a patient, an amount of time the fluid is to remain in the patient's peritoneal cavity (i.e., a dwell time), and an amount (or rate) of used dialysis fluid and ultrafiltration (“UF”) that is to be pumped or drained from the patient after the dwell period expires. For a treatment with multiple cycles, the parameters may specify the fill, dwell, and drain amounts for each cycle and the total number of cycles to be performed during the course of a treatment (where one treatment is provided per day or separate treatments are provided during the daytime and during nighttime). In addition, the parameters may specify dates/times/days (e.g., a schedule) in which treatments are to be administered by the medical fluid delivery machine. Further, parameters of a prescribed therapy may specify a total volume of dialysis fluid to be administered for each treatment in addition to a concentration level of the dialysis fluid, such as a dextrose level. For an infusion therapy, the parameters may include a volume to be infused, a medication to be infused, a medication concentration, a medication dosage, and/or an infusion rate.

Medical device data also includes data generated by a medical device that is indicative of measured, detected, or determined parameter values. The parameters for a treatment data may include, for example, a total amount of dialysis fluid administered to the patient, blood flowrate, dialysis fluid flowrate, dialysis dose, replacement fluid flow rate, used dialysis fluid for effluent flowrate, anticoagulant, e.g., citrate or heparin flowrate, calcium flowrate, dialysis fluid temperature, an intravenous drug flowrate, a number of cycles operated, a fill amount per cycle, a dwell time per cycle, a drain time/amount per cycle, an estimated amount of UF removed, a treatment start time/date, and/or a treatment end time/date. The treatment data may be prescribed or calculated, for example, such as a fill rate and a drain rate, determined by dividing the amount of fluid pumped by the time spent pumping.

Reference is further made herein to events and alarms. Events relate to administration of a treatment by a medical device and includes information indicative of the event and when the event occurred. The event may correspond to operator button presses, such as to pause a treatment. The event may also identify operations performed by a medical device, such as starting a treatment, ending a treatment, or changes made to a parameter during a treatment.

Alarms are indicative of fault information. Alarm information may include an identification of an alarm that occurred during a treatment, a duration of the alarm, a time of the alarm, an event associated with the alarm, and/or an indication as to whether the issue that caused the alarm was resolved or whether the alarm was silenced. The alarms may be related to a treatment, such as an alarm triggered when a treatment parameter exceeds an allowed limit or an alarm triggered after detection of a line occlusion. Alarms may also relate to diagnostics or operation of a medical device and include information indicative of internal operations of a medical device, such as faults related to pump operation, signal errors, communication errors, software issues, etc.

### I. Medical Device Environment Embodiment

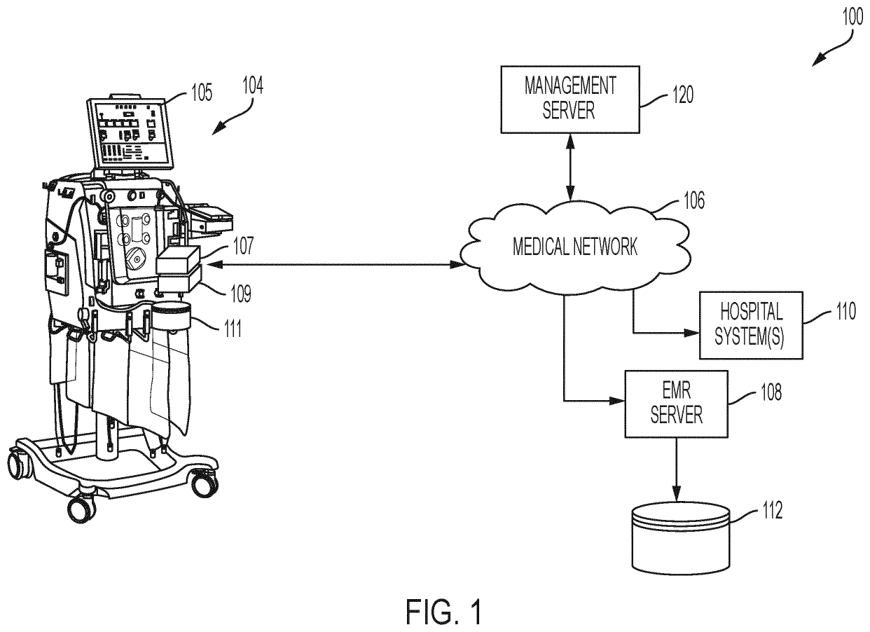

FIG. 1 is a diagram of a medical device environment 100, according to an example embodiment of the present disclosure. The example medical device environment 100 includes at least one medical device 104. While only one medical device 104 is shown in FIG. 1, it should be appreciated that the environment 100 may include tens to hundreds or thousands of medical devices.

The example medical device 104 is configured to accept one or more parameter specifying a treatment or prescription (i.e., treatment programming information). During operation, the medical device 104 writes alarm, event, diagnostic, and/or medical device data to one or more circular buffer at a rate between 1 Hz and 100 Hz and/or asynchronously. The data written to the circular buffers at this relatively fast rate is referred to herein as high resolution or high fidelity medical device data. Additionally, in some embodiments, the medical device 104 may store low resolution medical device data to a log file periodically, such as every five seconds to sixty seconds and/or after there is a change in data.

The example medical device 104 may include one or more control interfaces 105 for displaying instructions and receiving control inputs from a user. The control interface 105 may include buttons, a control panel, or a touchscreen. The control interface 105 may also be configured to enable a user to navigate to a certain window or user interface on a screen of the medical device 104. The control interface 105 may also provide instructions for operating or controlling the medical device 104.

The example medical device 104 also includes a processor or therapy module 107. The processor or therapy module 107 of the medical device 104 operates according to one or more instruction for performing a treatment on a patient. The instructions may be acquired via the control interface 105. The processor or therapy module 107 also monitors devices components for issues, which are documented as diagnostic information. The processor or therapy module 107 creates medical device data in combination with operating one or more pump or other component to administer the treatment. The therapy module 107 may also receive medical device sensor data from one or more communicatively coupled sensors (not shown).

The example medical device 104 further includes a control module or control processor 109 having at least one control processor and a memory device 111. The control processor 109 is configured to receive alarms, events, and high fidelity or high resolution medical device data from the therapy module 107. The control processor 109 determines if more than one circular buffer is present in the memory device 111. If one circular buffer is present, the control processor 109 stores the received medical device data to the circular buffer. However, if more than one circular buffer is present, the control processor 109 uses one or more rules to determine which subsets of the medical device data are to be stored to each of the circular buffers in the memory device 111. The example memory device 111 may include random access memory (“RAM”), read only memory (“ROM”), flash memory, magnetic or optical disks, optical memory, or other storage media for the circular buffer(s).

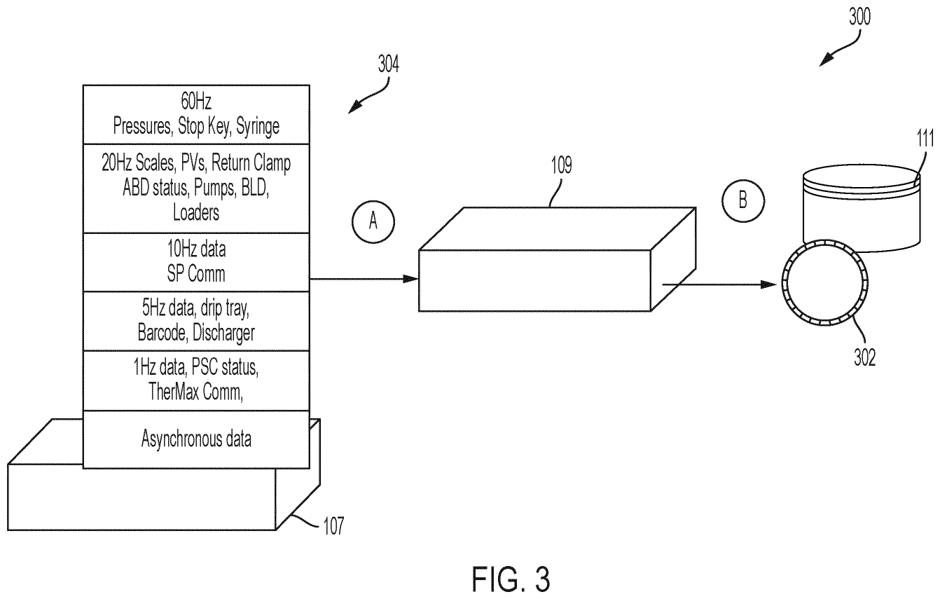

The example control processor 109 is configured to compare one or more alarm or event information to one or more conditions. As discussed herein, the conditions specify when medical device data in a circular buffer is to be retained or saved. If more than one circular buffer is configured, the conditions may assign or associate certain events/alarms to each of the circular buffers. Further, each circular buffer may only be configured to store a subset of the received medical device data. After detecting a condition is satisfied, the control processor 109 identifies the corresponding circular buffer in the memory 111 and creates a record or a file of the stored medical device data (or subset thereof). The record or file is transmitted to, for example, a management server 120, the hospital system 110, and/or stored in the memory device 111. Additional details regarding the control processor 109 and the circular buffers of the memory device 111 are discussed in more detail below in connection with FIGS. 3 to 5.

The example medical device environment 100 also includes a medical network 106, which communicatively couples the medical device 104 to an electronic medical record (“EMR”) server 108 and one or more hospital systems 110. The medical network 106 can include any number of gateways, routers, system hubs, switches, and/or network appliances for establishing communication connections and routing data. The medical network 106 may include one or more firewalls that restrict access to only authorized remote devices and/or servers. The medical network 106 may include any local area network (“LAN”), wireless LAN (“WLAN”), Ethernet network, Wi-Fi network, or combinations thereof.

As shown in FIG. 1, the medical device 104 may be wired or wirelessly coupled to the medical network 106. In some embodiments, the connection may include an Ethernet connection, a Wi-Fi connection, and/or a cellular connection. Additionally or alternatively, the medical device 104 may have a serial connection to the EMR server 108 (or the hospital system 110) that bypasses the medical network 106.

The example EMR server 108 of FIG. 1 is configured to manage patient EMRs that are stored in a database of a memory device 112. The EMR server 108 is configured to receive medical device data, parse the data based on patient identifier, locate corresponding patient EMRs in the memory device 112, and store the parsed medical device data to the identified EMR. The EMR server 108 may also access one or more EMR in response to request messages that identify the respective patients. The EMR server 108 may store the medical device data in a HL7 format, a binary version 2 format, a binary version 3 format, or a Fast Healthcare Interoperability Resources (“FHIR”) format.

The example hospital system 110 may include any of a service portal, an enterprise resource planning system, a web portal, a business intelligence portal, a HIPAA compliant database, a pharmacy system, etc. The hospital system 110 may also include a middleware system and/or an integration engine. The hospital system 110 enables user devices (e.g., smartphones, laptop computers, workstations, tablet computers, etc.) to read and/or write to the medical device data stored in the EMRs of the memory device 112.

In some embodiments, the control processor 109 of the medical device 104 may transmit buffered medical device data to a management server 120 via the medical network 106 and/or an external network. The management server 120 may include one or more models for modeling the received data to identify and/or diagnose a cause of an alarm or an event. The management server 120 may also create updated models and recommendations for local alarm/event diagnosis by the control module at the medical device 104.

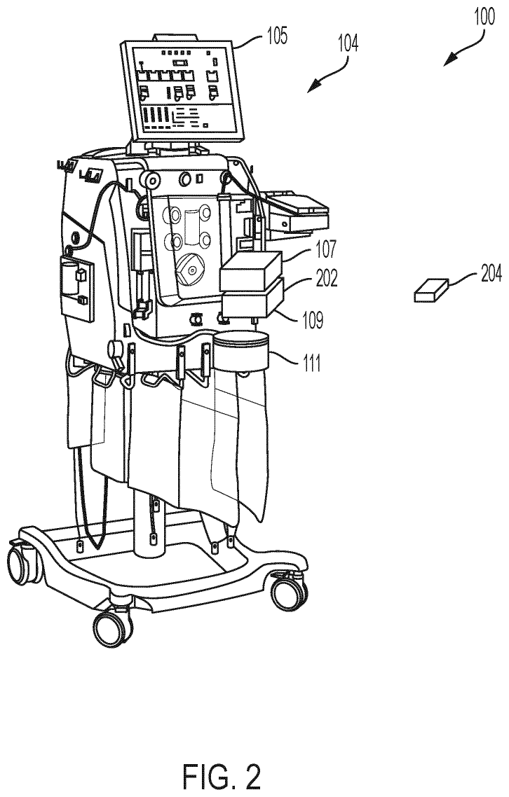

FIG. 2 is another diagram of the medical device environment 100, according to an example embodiment of the present disclosure. In this example, the control processor 109 includes a slot, hardware interface, or a port 202 for connection with a portable memory device 204. The slot or port 202 may be configured as a USB port, a Lightning® port, a micro USB port, an SD port, etc. The portable memory device 204 may include a USB-enabled memory device, an SD card, or other memory device that is removable from the port 202. Instead of transmitting medical device data from a circular buffer of the memory device 111, the control processor 109 is configured to copy or transfer the medical device data of the circular buffer to the portable memory device 204 after insertion. In some instances, the control processor 109 may copy or transfer records of medical device data and corresponding identifiers of alarms or events that trigged the creation of the record.

In the illustrated example, the medical device 104 of FIGS. 1 and 2 is the PrisMax™ CRRT machine manufactured by Baxter International Inc. It should be appreciated that in other embodiments, the medical device 104 may include any other renal failure therapy machine, infusion pump, physiological sensor, etc. The medical device 104 may include, for example, an infusion pump (e.g., a syringe pump, a linear peristaltic pump, a large volume pump (“LVP”), an ambulatory pump, multi-channel pump), a nutritional compounding machine, an oxygen sensor, a respiratory monitor, a glucose meter, a blood pressure monitor, an electrocardiogram (“ECG”) monitor, a weight scale, and/or a heart rate monitor.

Regarding renal failure therapy machines, due to various causes, a patient's renal system can fail. Renal failure produces several physiological derangements. For instance, a patient experiencing renal failure can no longer balance water and minerals or excrete daily metabolic load. Toxic end products of nitrogen metabolism (urea, creatinine, uric acid, and others) can accumulate in the patient's blood and tissue. Kidney failure and reduced kidney function have been treated with dialysis. Dialysis removes waste, toxins and excess water from the body that normal functioning kidneys would otherwise remove. Dialysis treatment for replacement of kidney functions is critical to many people because the treatment is life saving.

One type of kidney failure therapy is Hemodialysis (“HD”), which in general uses diffusion to remove waste products from a patient's blood. A diffusive gradient occurs across the semi-permeable dialyzer between the blood and an electrolyte solution called dialysate or dialysis fluid to cause diffusion.

Hemofiltration (“HF”) is an alternative renal replacement therapy that relies on a convective transport of toxins from the patient's blood. HF is accomplished by adding substitution or replacement fluid to the extracorporeal circuit during treatment (typically ten to ninety liters of such fluid). The substitution fluid and the fluid accumulated by the patient in between treatments is ultrafiltered over the course of the HF treatment, providing a convective transport mechanism that is particularly beneficial in removing middle and large molecules (in hemodialysis there is a small amount of waste removed along with the fluid gained between dialysis sessions, however, the solute drag from the removal of that ultrafiltrate is not enough to provide convective clearance).

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is provided directly to the extracorporeal circuit, providing convective clearance.

Another type of kidney failure therapy is peritoneal dialysis, which infuses a dialysis solution, also called dialysis fluid, into a patient's peritoneal cavity via a catheter. The dialysis fluid contacts the peritoneal membrane of the peritoneal cavity. Waste, toxins and excess water pass from the patient's bloodstream, through the peritoneal membrane and into the dialysis fluid due to diffusion and osmosis, i.e., an osmotic gradient occurs across the membrane. An osmotic agent in dialysis provides the osmotic gradient. The used or spent dialysis fluid is drained from the patient, removing waste, toxins and excess water from the patient. This cycle is repeated, e.g., multiple times.

There are various types of peritoneal dialysis therapies, including continuous ambulatory peritoneal dialysis (“CAPD”), automated peritoneal dialysis (“APD”), and tidal flow dialysis and continuous flow peritoneal dialysis (“CFPD”). CAPD is a manual dialysis treatment. Here, the patient manually connects an implanted catheter to a drain to allow used or spent dialysate fluid to drain from the peritoneal cavity. The patient then connects the catheter to a bag of fresh dialysis fluid to infuse fresh dialysis fluid through the catheter and into the patient. The patient disconnects the catheter from the fresh dialysis fluid bag and allows the dialysis fluid to dwell within the peritoneal cavity, wherein the transfer of waste, toxins and excess water takes place. After a dwell period, the patient repeats the manual dialysis procedure, for example, four times per day, each treatment lasting about an hour. Manual peritoneal dialysis requires a significant amount of time and effort from the patient, leaving ample room for improvement.

Automated peritoneal dialysis (“APD”) is similar to CAPD in that the dialysis treatment includes drain, fill and dwell cycles. APD machines, however, perform the cycles automatically, typically while the patient sleeps. APD machines free patients from having to perform the treatment cycles manually and from having to transport supplies during the day. APD machines connect fluidly to an implanted catheter, to a source or bag of fresh dialysis fluid and to a fluid drain. APD machines pump fresh dialysis fluid from a dialysis fluid source, through the catheter and into the patient's peritoneal cavity. APD machines also allow for the dialysis fluid to dwell within the cavity and for the transfer of waste, toxins and excess water to take place. The source may include multiple sterile dialysis fluid bags.

APD machines pump used or spent dialysate from the peritoneal cavity, though the catheter, and to the drain. As with the manual process, several drain, fill and dwell cycles occur during dialysis. A “last fill” occurs at the end of APD and remains in the peritoneal cavity of the patient until the next treatment.

The present system and associated methodology are applicable to any of the above renal failure therapy modalities.

### II. Circular Buffer Embodiment

In the subsequent sections, reference is made to operations performed by control processor 109 of the medical device 104. The example operations performed by the control processor 109 may be implemented using one or more computer program and/or application. The programs or the applications may be defined by a series of computer instructions that are stored on any computer-readable medium, including random access memory (“RAM”), read only memory (“ROM”), flash memory, magnetic or optical disks, optical memory, or other storage media. The instructions may be configured to be executed by control processor 109, which when executing the series of computer instructions performs or facilitates the performance of all or part of the disclosed methods and procedures disclosed herein. The persistent storage device may include any memory device including RAM, ROM, flash memory, etc.

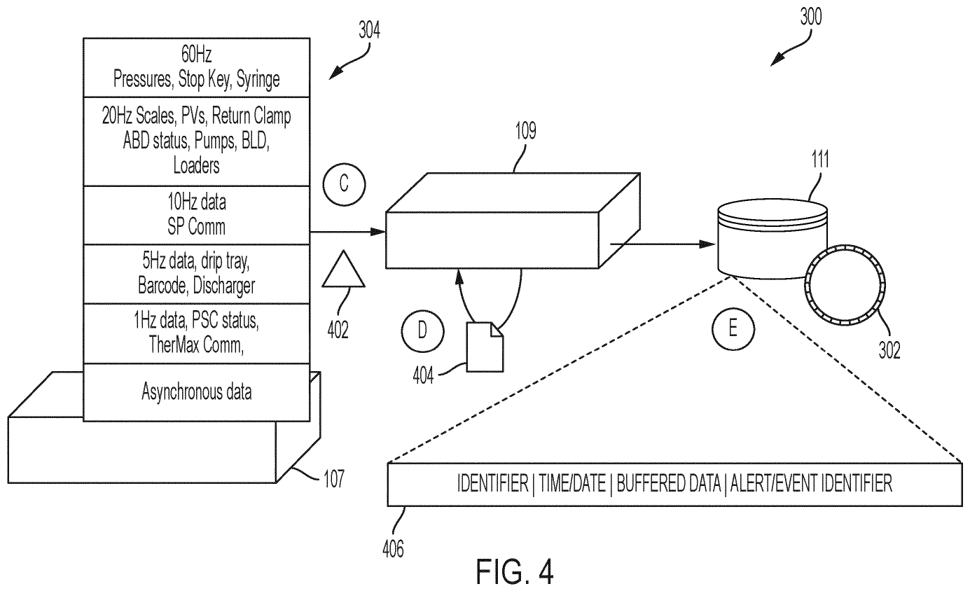

FIGS. 3 and 4 are diagrams of an example procedure for using a circular buffer 302 in the memory device 111 of FIGS. 1 and 2 for creating records or files of medical device data that relates to an event or an alarm, according to an example embodiment of the present disclosure. As shown in FIG. 3 at Event A, a therapy operations processor of a therapy module 107 within the medical device 104 generates medical device data 304. The therapy module 107 transmits the medical device data 304 to the control processor 109.

In the illustrated example, the medical device data 304 includes pressure measurement data, stop key press indications, and syringe position information, which are generated at a rate of 60 Hz. The medical device data 304 also includes scale measurements, pinch valve data, clamp status, air bubble detection (“ABD”) status, pump status/rate data, blood rate detection data, and loader status, which is generated at a rate of 20 Hz. The medical device data 304 further includes inter-processor communication status data (e.g., status of communication with a safety processor), which is generated at a rate of 10 Hz. Additionally, the medical device data 304 may include drip tray data (for detecting a line leak), barcode read data, and discharger status, which are generated at 5 Hz. Moreover, the medical device data 304 includes a status of a power supply controller (e.g., battery health status) and communication status of the medical device 104, which is generated at a rate of 1 Hz. Finally, the medical device data 304 includes a subset of data that is generated asynchronously. This may include the generation of alarms or events. It should be appreciated that in other embodiments, additional medical device data may be generated or less medical device data may be generated. Further, the data may be generated at different rates than those discussed above.

As shown, the medical device data 304 is generated as a stream of data that is transmitted to the control processor 109 at the rates described above. After receiving the medical device data 304, at Event B, the control processor 109 writes at least some of the data 304 to the circular buffer 302 of the memory device 111. In some instances, the control processor 109 is programmed with one or more rules that define a subset of the medical device data 304 to be written to the circular buffer 302. For example, a rule may specify that all of the 60 Hz, 20 Hz, 5 Hz, and asynchronous medical device data 304 is to be written to the circular buffer 302. The rules may specify data fields, data position, and data identifiers to enable the control processor 109 locate the appropriate medical device data 304 within the stream from the therapy module 107. In this example, the control processor 309 may disregard the 10 Hz and 1 Hz medical device data 304. In other instances, the control processor 109 may write all of the received medical device data 304 to the circular buffer 302.

During Event B (and subsequent Events C to E), the therapy module 107 continues to transmit a stream of the medical device data 304 at the specified data transmission rate. During this time, the control processor 109 is configured to first fill the circular buffer 302 until the allocated memory space is full. The allocated memory space may correspond to a predetermined time duration of data, such as a time duration between ten seconds to two minutes. After the circular buffer 302 is filled, the control processor 109 overwrites the relatively oldest medical device data 304 with the current medical device data 304 such that the circular buffer 302 contains the most recent medical device data of the designated time duration.

The example procedure 300 continues in FIG. 4. At Event C, the therapy module 107 generates an alarm or an event, which is indicated via message 402. The control processor 109 receives the alarm or event message 402 with the medical device data 304. The alarm or event message 402 may include an identifier and/or a time/date of the alarm generation that is used by the control processor 109 to determine which alarm or event was generated. Next, at Event D, the control processor 109 determines if the alarm or event message 402 matches one or more conditions specified in a rule set 404. The example rule set 404 may be stored in the memory device 111 (or another memory device) and specifies conditions for which a record 406 is to be created with the buffered medical device data 304. The conditions may identify certain alarm or event types. For instance, upon detecting a blood leak detection alarm, the rule set 404 specifies that the control processor 109 is to create a record 406 using the medical device data 304 located in the circular buffer 302. Other conditions may include identification of an occlusion alarm, a pressure alarm, a low fluid volume alarm, a flow rate alarm, a syringe alarm, a treatment pause event, a treatment stoppage event, a treatment parameter change event, etc.

The conditions may also specify medical device data threshold values. For example, after determining that a fluid pressure exceeds a threshold, the rule set 404 specifies that the control processor 109 is to create a record 406 using the medical device data 304 located in the circular buffer 302. The conditions may also specify data change rates of the medical device data 304, or data derivatives/computations specified by an operator.

If at Event D the control processor 109 determines that a record 406 is to be created based on the detected alarm or event message 402, the control processor 109 at Event E reads the medical device data 304 that is located in the circular buffer 302 of the memory device 111. The control processor 109 stores the read data to a record 406 (e.g., a single data package). The control processor 109 stores the record 406 to a machine log storage area with a filename that is uniquely created based on the individual alarm or event message 402 that occurred. In some embodiments, the record 406 includes a unique identifier, an identifier of the alarm or the event message 402, a time/date the alarm or event message 402 was generated/received, and the read medical device data 304 from the circular buffer 302. In some instances, the unique identifier may correspond to a time/date stamp of a beginning of a treatment and/or a time/date stamp of an end of a treatment. Additionally or alternatively, the record 406 may be stored to a section of the machine log that identifies time/date stamp of a beginning of a treatment and/or a time/date stamp of an end of a treatment. The example procedure 300 then proceeds for the next alarm or event generated.

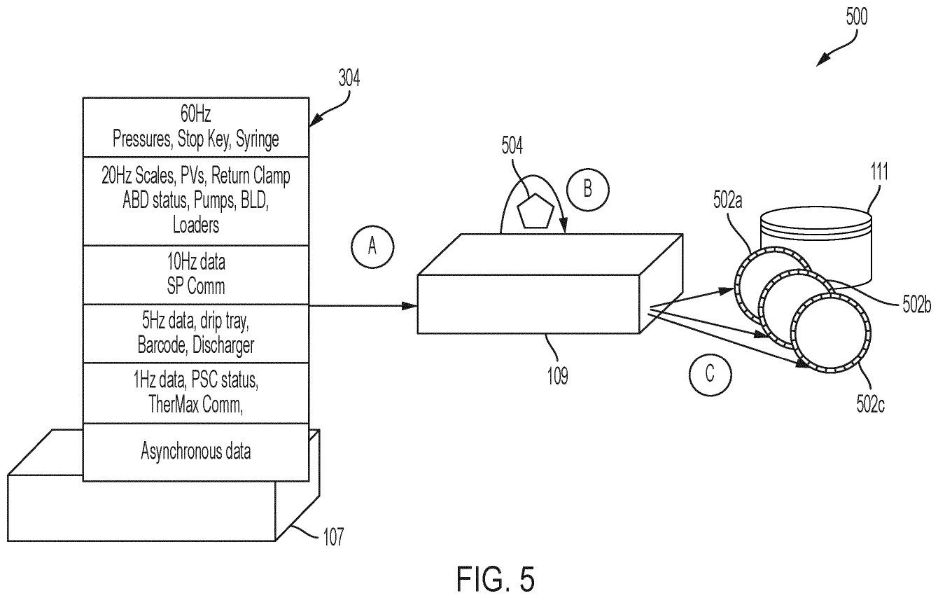

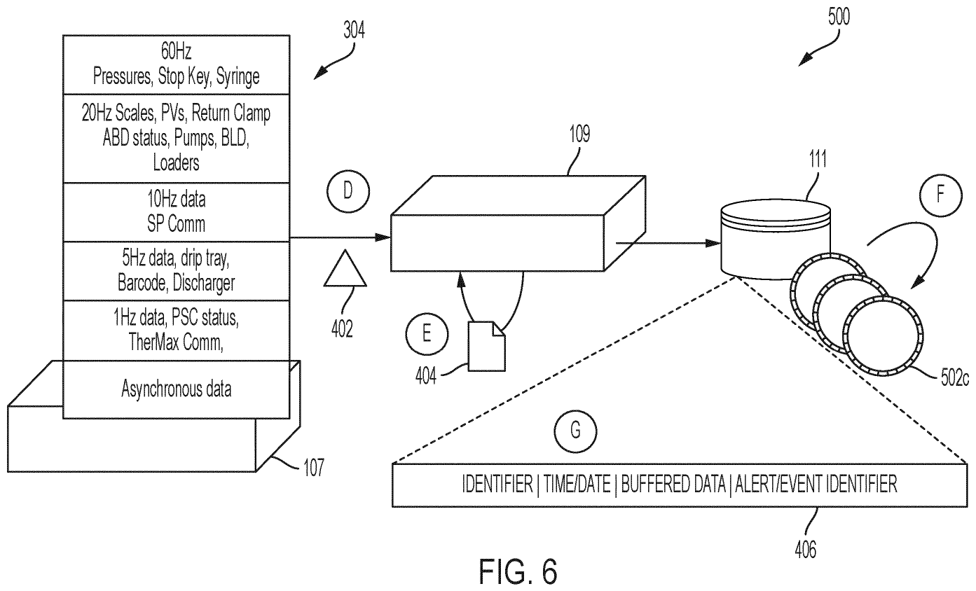

FIGS. 5 and 6 are diagrams of an example procedure for using multiple circular buffers 502a, 502b, and 502c in the memory device 111 of FIGS. 1 and 2 for creating records or files of medical device data that relates to an event or an alarm, according to an example embodiment of the present disclosure. It should be appreciated that only three circular buffers 502 are shown for brevity. The memory device 111 may contain a circular buffer 502 for each of the medical device data rates. For example, circular buffer 502a may be designated for storing the medical device data 304 that is generated at a 60 Hz rate while circular buffer 502b may be designated for storing medical device data 304 that is generated at a 20 Hz rate and circular buffer 502c may be designated for storing medical device data 304 that is generated at a 10 Hz rate.

In some instances, each of the circular buffers 502 is configured to store a same time duration of medical device data 304. In these instances, the circular buffer 502a for the 60 Hz subset of medical device data 304 would likely store significantly more data compared to the circular buffer for the 10 Hz medical device data since data is generated at a faster 60 Hz rate. In other instances, each of the circular buffers 502 has a different specified time duration (i.e., allocated memory size) that provides for a minimal amount of data needed for diagnosis. In these other instances, the circular buffer 502a may have a shorter time duration of twenty seconds since data is stored at a faster rate, while the circular buffer 502c may be configured to store sixty seconds of medical device data generated at 10 Hz. The time duration may account for the minimal threshold in addition to an additional time guard band to ensure a sufficient amount of data is retained.

In other embodiments, the circular buffers 502 are configured based on alarm or event type. For example, the circular buffer 502a may be configured to store only a subset of the medical device data 304 that is relevant or has a first and/or second order relation to a pressure alarm. Thus, when a pressure alarm is detected, the control processor 109 inly has to read the circular buffer 502a. A rule set 504 may define to which circular buffer 502 certain subsets of the medical device data 304 is to be written. The rule set 504 may identify data fields and/or data identifiers and the corresponding circular buffer 502. The rule set 504 may also be used for identifying which medical device data 304 is associated with a certain data rate for writing to the appropriate circular buffer 502.

As shown in the procedure 500 of FIG. 5, at Event A, the therapy module 107 generates and transmits the medical device data 304 to the control processor 109. At Event B, the control processor 109 uses the rule set 504 to identify and determine which of the medical device data 304 is to be written to each of the circular buffers 502. At Event C, the control processor 109 writes the subsets of medical device data 304 to the appropriate circular buffer 502. As described above, the rule set 504 may identify which of the data 304 corresponds to a data rate, and identifies the corresponding circular buffer 502. In this example, the control processor 109 writes (and overwrites) the 60 Hz subset of the medical device data 304 to the circular buffer 502a.

The example procedure 500 continues in FIG. 6. At Event D, the therapy module 107 generates an alarm or an event message 402. The control processor 109 receives the alarm or event message 402 with the medical device data 304. Next, at Event E, the control processor 109 determines if the alarm or event message 402 matches one or more conditions specified in a rule set 404. As described above, the example rule set 404 may be stored in the memory device 111 and specifies conditions for which a record 406 is to be created with the buffered medical device data 304.

If at Event E the control processor 109 determines that a record 406 is to be created based on the detected alarm or event message 402, the control processor 109 at Event F reads the medical device data 304 that is located in the corresponding circular buffer 502. For example, the control processor 109 selects the circular buffer 502 with the data 304 that corresponds to the received alarm or event message 402. In some instances where data is stored in the circular buffers based on data rate, the control processor 109 reads all of the circular buffers 502 of the memory device 111.

At Event G, the control processor 109 stores the read data to a record 406 (e.g., a single data package). The control processor 109 stores the record 406 to a machine log storage area with a filename that is uniquely created based on the individual alarm or event message 402 that occurred. The record 406 includes, for example, a unique identifier, an identifier of the alarm or the event message 402, a time/date the alarm or event message 402 was generated/received, and the read medical device data 304 from the circular buffer 302. In some instances, the unique identifier may correspond to a time/date stamp of a beginning of a treatment and/or a time/date stamp of an end of a treatment. Additionally or alternatively, the record 406 may be stored to a section of the machine log that identifies time/date stamp of a beginning of a treatment and/or a time/date stamp of an end of a treatment. The example procedure 500 then proceeds for the next alarm or event generated.

### III. Medical Device Diagnosis Embodiment

In some embodiments, the control processor 109 and/or the management server 120 of FIG. 1 are configured to display a replay of conditions on the medical device 104 using the medical device data contained in one or more records or files. In an example, for a given alarm or event, the control processor 109 and/or the management server 120 accesses a corresponding model (e.g., a Matlab® model). For instance, a pressure model may exist for a pressure alarm. Each model processes the medical device data 304 so as to determine a relationship as to when the alarm or event occurred and changes in the data. The models may provide a simulation of the medical device 104 for that specific alarm or event using the medical device data of the record to show a state change of the medical device 104 leading to the alarm or event. Additionally or alternatively, the models may provide a plot each of the different types of medical device data 304 on respective time-series graphs that covers the time duration of the respective circular buffer. In some instances, the models may define medical device data types that have at least a known first order relationship or a known second order relationship with the alarm or event.

Using the records, the graphs or models have enough medical device data history and resolution for the identification of any significant changes leading up to the generation of the alarm or the event. If the record contains data after the event or alarm, the control processor 109 causes this additional data to be displayed as well. The control processor 109 may also use a date/time stamp related to the alarm or the event to provide an indication on the time-series graphs that is indicative of when the alarm or event was generated, thereby providing a relation between the data and the alarm/event.

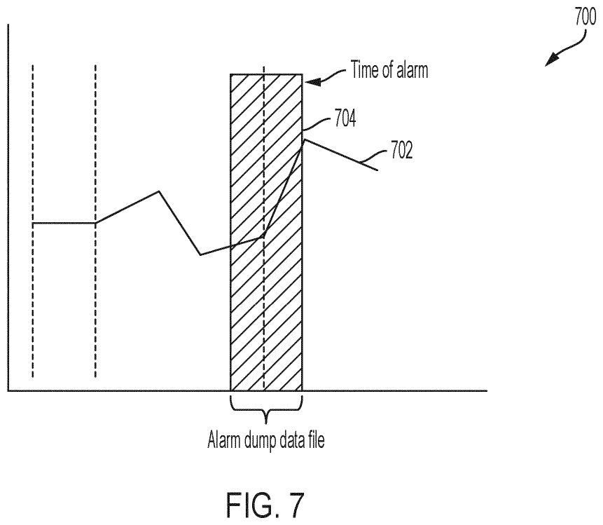

FIG. 7 is a diagram of a graph 700 that is illustrative of medical device data 304 stored in a circular buffer and written to a record for diagnosis of an event or alarm, according to an example embodiment of the present disclosure. In the graph 700, line 702 represents a blood line pressure measurement, which is generated by the therapy module 107 using sensed blood pressure. Point 704 represents that a blood pressure alarm was generated. A portion of the line 702 that is within the hashed box corresponds to the amount of the pressure data that is stored in a circular buffer and written to a record associated with the alarm. As shown, the amount of recorded data includes a portion of the line 702 that begins to spike. This spike may be analyzed as a root cause for the alarm, which may indicate a clot of a dialyzer or blood filter, which has caused the pressure to increase.

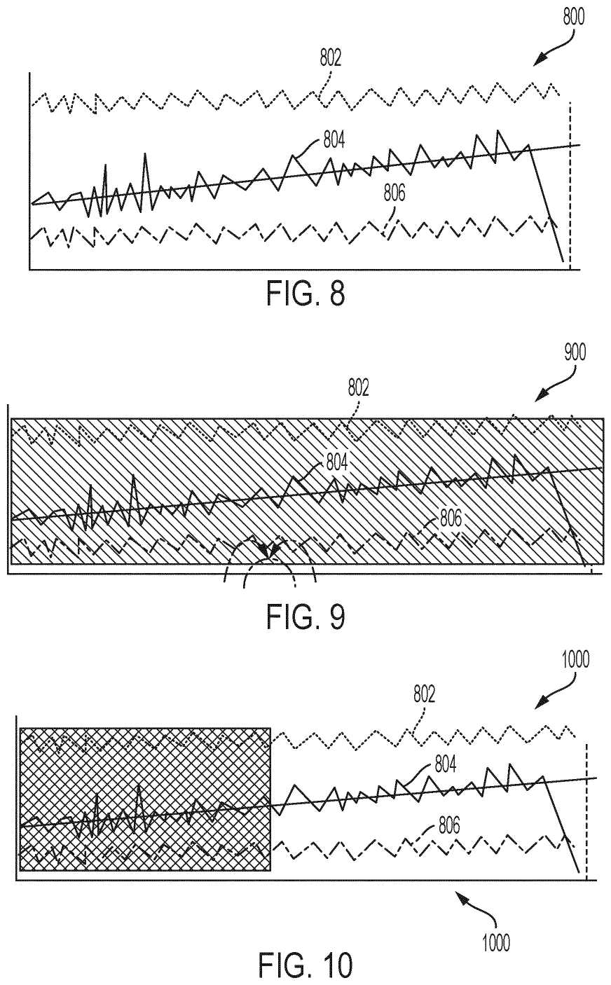

FIG. 8 is a diagram of a graph 800 that is illustrative of medical device data 304 generated by the medical device 104 of FIGS. 1 and 2, according to an example embodiment of the present disclosure. In this example, data line 802 may corresponds to dialysis line pressure, line 804 corresponds to blood line pressure, and line 806 corresponds to dialysis flow rate. As shown, a blood pressure change coincides with a generation of an alarm or event. The change of the blood pressure while the dialysis fluid pressure and dialysis flow rate did not change provides an indication of a cause of the alarm or the alert, such as a blood line disconnection.

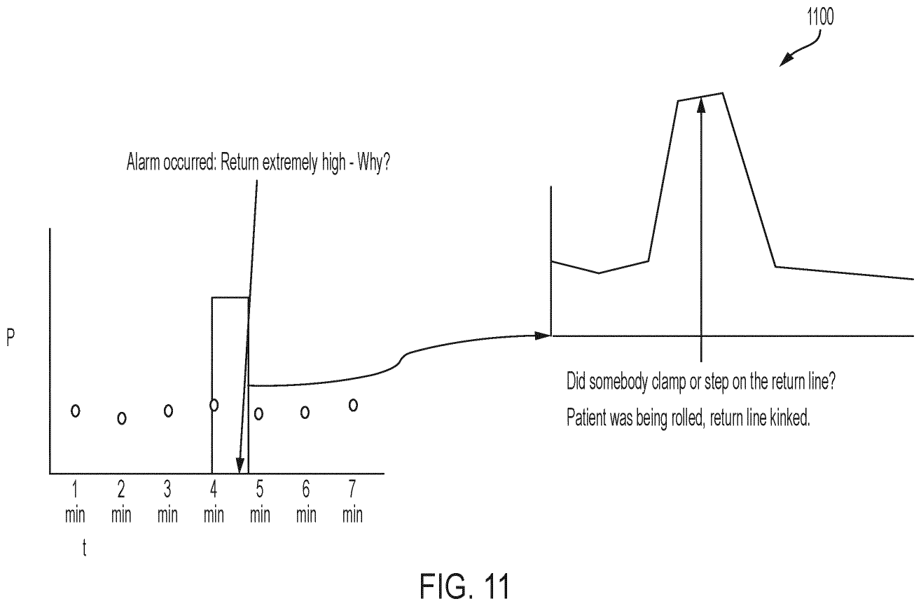

FIG. 9 is a diagram of a graph 900 that shows which data is available without the use of the circular buffers. As shown, the history of the data is not available since it did not occur during a time interval in which the data was sampled. As a result, a root cause for the alarm may not be able to be determined.

In contrast FIG. 10 is a diagram of a graph 1000 that shows which portion of the medical device data 304 is available with the circular buffers, according to an example embodiment of the present disclosure. As shown, most recent medical device data 304, shown outside of the hashed box, is stored in the circular buffers. After activation of the alarm or event, this data is written to a record for diagnosis. The specified time duration is sufficient to provide enough data history to capture the change in the blood line pressure, shown in line 804. However, the vast amount of data that occurred minutes to hours before the alarm is not relevant and accordingly not retained through the use of the circular buffers.

FIG. 11 is a diagram of a user interface 1100 with diagnosis information provided by the control processor 109 and/or the management server 120 of FIG. 1, according to an example embodiment of the present disclosure. The user interface 1100 shows an occurrence of an alarm indicative that a pressure on a blood return line exceeds a threshold. The user interface 1100 also shows medical device data 304 including blood pressure measurements for the blood return line. In this example, the control processor 109 and/or the management server 120 determined of all of the buffered medical device data 304, the return blood measurement had a change in rate that corresponded to the occurrence of the alarm.

The user interface 1100 also provides potential causes of the alarm, such as a line clamp or kink. In some instances, the control processor 109 and/or the management server 120 identifies or analyzes line slope, derivative data, area under a curve, data point average, etc. for identifying a cause and providing a recommendation. The control processor 109 and/or the management server 120 may also use template matching to known causes or machine learning algorithms to determine a cause for an alarm or alert.

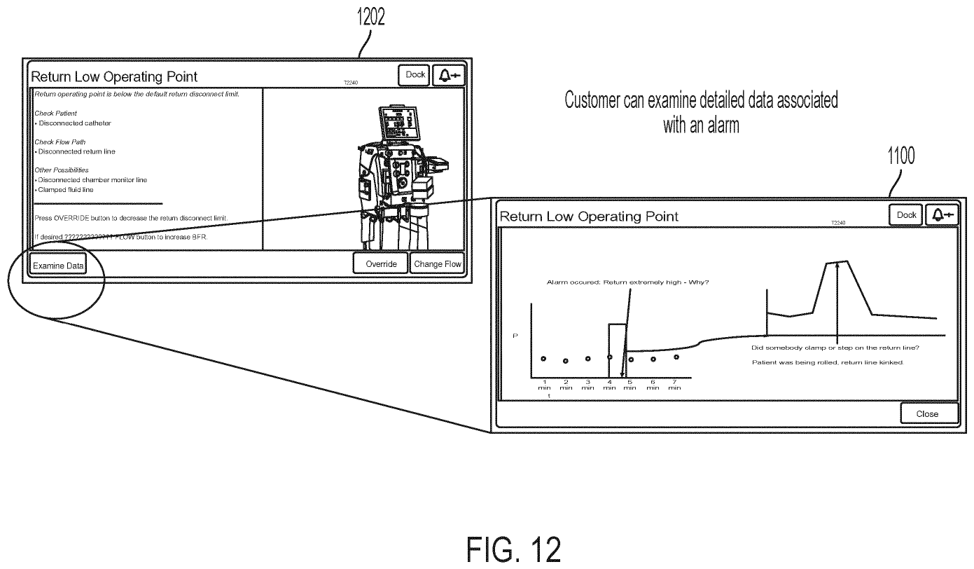

FIG. 12 is a diagram showing that the user interface 1100 of FIG. 11 may be generated by the control processor 109 of the medical device 104 after selection of an “Examine Data” feature on an alarm mediation interface 1202, according to an example embodiment of the present disclosure. In this example, the user interface 1202 is displayed to show possible causes of an alarm. Selection of the “Examine Data” feature causes the control processor 109 to display the user interface 1100. In some instances, the control processor 109 may analyze all of the medical device data in the record related to the alarm to identify which data changed just prior to activation of the alarm. In these instances, the control processor 109 displays the identified medical device data. In other instances, the control processor 109 displays in a time-series graph all of the medical device data stored in the record to enable an operator to determine which data may be related to the generation of the alarm.

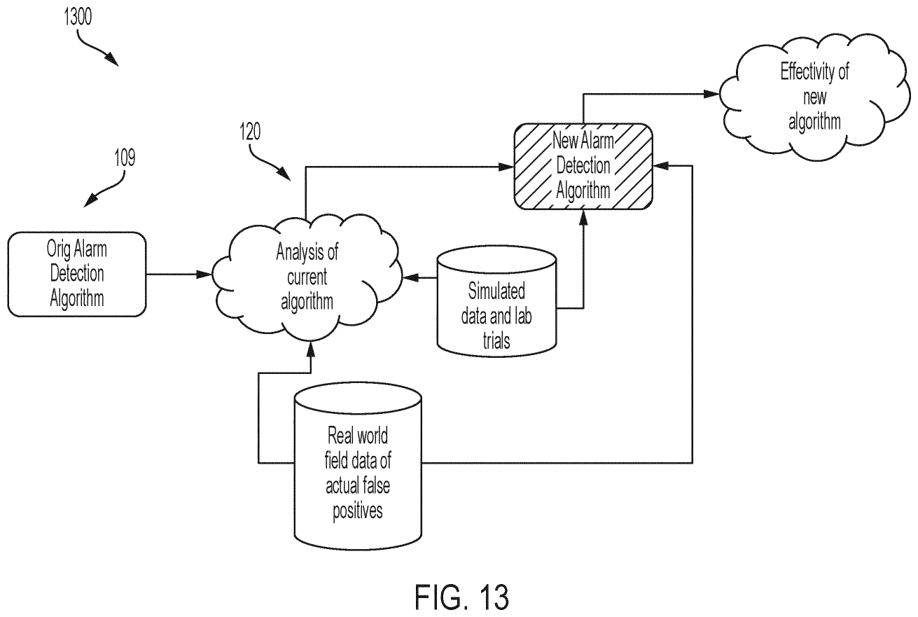

In some embodiments, the management server 120 is configured to simulate operation of the medical device 104 using one or more models that related to generated alarms and/or events. FIG. 13 is a diagram of an example process 1300 for simulating operation of a medical device 104 using one or more models that related to generated alarms and/or events. The server 120 receives one or more records from the control processor 109. The server 120 then populates the medical device data into the models to provide a replay of the operation of the medical device 104. An operator can view how the medical device's operation changed overtime to determine which factors may have contributed to an alarm and/or an event.

The simulation may also enable developers to test new limits, thresholds, or conditions for generating alarms and/or events, or new alarm detection algorithms. For example, after setting a new blood pressure alarm limit to reduce an occurrence of false-alarms, the developer may perform simulations of the medical device 104 with the new limit using medical device data 304 from records that are related to the alarm being tested. The developer may then determine if/when alarms would be generated using the new limits using the simulations to determine an effectivity of the new algorithm/limits, and fine-tune the algorithm/alarm limits as necessary until the desired response is achieved.

In some embodiments, the medical device is a PD machine that is configured to perform a PD treatment on a patient. In these embodiments, the control processor 109 is configured to store to one or more circular buffers of a memory device PD-related medical device data. The PD-related medical device data may include a fill rate at which dialysis fluid is pumped into a patient's peritoneal cavity, a drain rate at which the dialysis fluid is removed from the patient's peritoneal cavity, a dwell time, an estimated amount of UF removed from the patient, an estimated residual volume of fluid within a patient, a dextrose concentration of a dialysis fluid provided to a patient, and/or physiological sensor measurements, such as a pressure measurement corresponding to a patient's peritoneal cavity pressure. Each of the above PD-related medical device data may be generated at different rates and stored to the one or more circular buffers of the PD machine accordingly.

In these PD-related embodiments, the control processor 109 and/or the management server 120 are configured to display a replay of conditions on the medical device 104 using the medical device data contained in one or more records or files. In an example, an alarm or an event condition may be generated due to a sudden increase in a patient's peritoneal cavity pressure during a fill phase of dialysis fluid. The high fidelity medical device data stored in a circular buffer shows a fill pump rate in relation to the measured cavity pressure, and an estimated fill volume (based on a residual volume of fluid from a previous fill-drain cycle). An analysis of the data at millisecond intervals may identify that the estimated patient fill volume was underestimated, which caused the patient to become overfilled with dialysis fluid. In another example, a sudden increase in line pressure to a patient may be indicative of an inadvertent line occlusion. As shown above, the use of the circular buffers in the PD machine enables alarms and events to be effectively analyzed to determine a root cause for improving a patient's treatment.

In another example, the control processor 109 causes certain pressure alarms to occur repeatedly. A user interface of the PD machine provides information for a recent circular buffer model related to the pressure alarms. For example, line pressure data may be displayed on a graph in addition to possible causes of the alarm and possible remedies. In this example, one listed possible cause indicates that the alarm may be due to occlusion of a return/access line as a result of the patient being re-positioned. The date/time of the displayed alarm data aligns with when the clinician knows the patient was re-positioned. The clinician inspects the return/access line and corrects for the occlusion. As the line pressure returns to a normal range, the control processor 109 ends the pressure alarms, allowing treatment to continue un-interrupted.

### IV. Conclusion

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1: A renal therapy apparatus comprising:
a memory device configured as a circular buffer;
a therapy operations processor configured to generate (i) alarms, (ii) events, and (iii) high fidelity medical device data, (i), (ii), and (iii) being related to operation of the renal therapy apparatus for performing a renal therapy treatment, the medical device data including at least two of first data generated at a 1 Hz rate, second data generated at a 5 Hz rate, third data generated at a 10 Hz rate, fourth data generated at a 20 Hz rate, fifth data generated at a 60 Hz rate, and sixth data generated at an asynchronous rate;
a control processor communicatively coupled to the memory device and the therapy operations processor, the control processor configured to
receive the medical device data at the specified data rates from the therapy operations processor,
store a first stream of the medical device data to the memory device in a circular buffer configuration such that previous medical device data stored to the memory device is overwritten after being in the memory device for a first specified time duration,
determine that at least one of an alarm or an event has been generated by the therapy operations processor,
store, for a second specified time duration after the occurrence of the at least one of the alarm or the event, a second stream of the medical device data to the memory device in the circular buffer configuration such that previous medical device data stored to the memory device is overwritten after being in the memory device for the first specified time period, and
after the second specified time duration has elapsed, create a file that includes the medical device data in the circular buffer and information indicative of the at least one of the generated alarm or event.

2: The apparatus of claim 1, wherein the medical device data includes at least one of pump rate data, pressure data, temperature data, scale data, or diagnostic data.

3: The apparatus of claim 1, wherein the first specified time duration is between ten seconds and two minutes and the second time duration is between zero seconds and thirty seconds.

4: The apparatus of claim 1, further comprising a port to receive a portable memory device,
wherein the control processor is configured to transmit the file to the portable memory device after detecting that the portable memory device is communicatively coupled to the port.

5: The apparatus of claim 1, wherein the control processor is configured to transmit the file to a server via a network after the file is created.

6: The apparatus of claim 1, wherein the file includes at least a portion of the first stream of the medical device data before the at least one of the alarm or the event and the second stream of the medical device data after the at least one of the alarm or the event to enable a server to recreate conditions of the renal therapy apparatus for identifying a cause of the at least one of the alarm or the event.

7: A medical device apparatus comprising:
a therapy operations processor configured to generate alarms, events, and high fidelity medical device data that are related to an operation of the medical device apparatus for performing a medical treatment;
a memory device including a first circular buffer configured to store a first duration of medical device data and a second circular buffer configured to store a second duration of medical device data, the first circular buffer configured to store a first subset of the medical device data and the second circular buffer configured to store a second subset of the medical device data;
a control processor communicatively coupled to the memory device and the therapy operations processor, the control processor configured to
receive a stream of medical device data from the therapy operations processor,
identify as a first stream, the first subset of the received medical device data,
identify as a second stream, the second subset of the received medical device data,
write the first stream to the first circular buffer such that a most recent first duration of the first stream is stored,
write the second stream to the second circular buffer such that a most recent second duration of the second stream is stored,
detect an occurrence of an alarm or event, and
create a record that includes an identifier of the alarm or event and at least the first subset of the medical device data that is stored in the first circular buffer.

8: The apparatus of claim 7, wherein the control processor is configured to additionally include in the record the second subset of the medical device data that is stored in the second circular buffer.

9: The apparatus of claim 7, wherein the first duration and the second duration have a same time duration.

10: The apparatus of claim 7, wherein the first duration has a time duration that is longer than the second duration.

11: The apparatus of claim 7, wherein the first duration and the second duration are each between ten seconds and two minutes.

12: The apparatus of claim 7, wherein the first subset of the medical device data is generated at a first data rate and the second subset of medical device data is generated at a second, different data rate.

13: The apparatus of claim 12, wherein the first data rate and the second data rate are each between 1 Hz and 100 Hz.

14: The apparatus of claim 12, wherein at least one of the first data rate or the second data rate includes an asynchronous data rate.

15: The apparatus of claim 7, wherein the detected alarm or event is a first alarm or event, and
wherein the first subset of the medical device data is associated with the first alarm or event and the second subset of the medical device data is associated with a second, different alarm or event.

16: The apparatus of claim 7, wherein the alarm or event includes at least one of an occlusion alarm, a pressure alarm, a low fluid volume alarm, a flow rate alarm, a syringe alarm, a fluid leak detection alarm, a blood leak detection alarm, an air bubble detection alarm, a power supply alarm, a treatment pause event, a treatment stoppage event, or a treatment parameter change event.

17: The apparatus of claim 7, wherein the control processor is configured to transmit the record via at least one of (i) a network to a server, or (ii) a port to a portable memory device for diagnosis of a cause of the alarm or event.

18: The apparatus of claim 7, wherein the control processor is configured to model or analyze at least the first subset of the medical device data that is included within the record for diagnosis of a cause of the alarm or event.

19: The apparatus of claim 7, wherein the control processor is configured to display on a display screen in a time-series graph at least some of the first subset of the medical device data included within the record.

20: The apparatus of claim 7, wherein the medical device apparatus includes at least one of a renal therapy machine or an infusion pump.

21: A medical device method comprising:
receiving, in a control processor of a medical device, a stream of medical device data;
identifying, via the control processor, as a first stream, a first subset of the received medical device data;
identifying, via the control processor, as a second stream, a second subset of the received medical device data;
writing, via the control processor, the first stream to a first circular buffer in a memory device such that a most recent first duration of the first stream is stored;
writing, via the control processor, the second stream to a second circular buffer in the memory device such that a most recent second duration of the second stream is stored;
receiving, in the control processor, an indication or an alarm or an event; and
creating, via the control processor, a record that includes an identifier of the alarm or event and at least the first subset of the medical device data that is stored in the first circular buffer.

22: The method of claim 21, wherein creating the record further comprises including the second subset of the medical device data that is stored in the second circular buffer.

23: The method of claim 21, wherein the first circular buffer is configured to receive medical device data that is generated at a first data rate and the second circular buffer is configured to receive medical device data that is generated at a second, different data rate.

24: The method of claim 23, wherein the first duration is different from the second duration and each of the first and second durations are between ten seconds and two minutes.
